(19) United States
(12) Patent Application Publication
(10) Pub. No.: US 2021/0386329 A1
(43) Pub. Date: Dec. 16, 2021

(54) MEASUREMENT DEVICE, CONTROL METHOD, AND PROGRAM RECORDING MEDIUM

(71) Applicant: NEC CORPORATION, Tokyo (JP)

(72) Inventors: Chenhui HUANG, Tokyo (JP); Kenichiro FUKUSHI, Tokyo (JP); Hiroshi KAJITANI, Tokyo (JP); Kentaro NAKAHARA, Tokyo (JP)

(73) Assignee: NEC Corporation, Tokyo (JP)

(21) Appl. No.: 17/283,295

(22) PCT Filed: Oct. 17, 2018
(86) PCT No.: PCT/JP2018/038698

§ 371 Date: Apr. 7, 2021

(51) Int. Cl. A61B 5/11

(52) U.S. Cl. CPC A61B 5/1123; A61B 5/112; A61B 5/1126; A61B 5/1118; A61B 5/1113

(57) ABSTRACT

A measurement device includes: a data acquisition unit that measures a sensor detection value by a sensor in at least two operation modes including a first mode with low power and a second mode for operating at a high speed, transmits a trigger signal when the detected value exceeds a first threshold value while operating in the first mode, transmits a first notification signal notifying that the user has started walking when the detected value has exceeded a second threshold value while operating in the second mode, and starts measuring gait data including a walking characteristic of the user wearing the sensor based on the sensor detection value; and a control unit that switches the operation mode to the second mode when the trigger signal is received, and switches the operation mode to the first mode when a predetermined condition is satisfied after reception of the first notification signal.

### TECHNICAL FIELD

The present invention relates to a measurement device, a measurement method, and a program that measure gait data of a user.

### BACKGROUND ART

In order to monitor the walking state of a user, a walking sensor worn on a foot has been developed. Such a walking sensor is often mounted with a small-sized battery having a small capacity in such a way that walking is not affected. Such a walking sensor consumes a great deal of power to perform measurements at all times and is difficult to use for a long time without changing or charging the battery. Therefore, a power-saving walking sensor is expected.

PTL 1 discloses a method for analyzing the motion of a foot relative to the ground. In the method of PTL 1, the acceleration of the foot is captured using an accelerometer, and the moment at which the foot leaves the ground is determined.

PTL 2 discloses a biometric information measurement system constituted by a biometric information detection apparatus that transmits biometric information data detected by a sensor via wireless communication, and a portable apparatus having a first identification code associated with a user in advance. The portable apparatus includes a detection apparatus specifying means for specifying the biometric information detection apparatus in use, an identification code creating means for creating a second identification code that identifies the biometric information detection apparatus, and an identification code transmitting means for wirelessly transmitting the second identification code to the biometric information detection apparatus in use.

### CITATION LIST

### Patent Literature

[PTL 1] JP 4448901 B2
[PTL 2] JP 4555596 B2

### SUMMARY OF INVENTION

### Technical Problem

According to the method of PTL 1, the sensor working time can be extended by measuring a time difference between each moment at which the foot touches the ground and a following moment at which the foot leaves the ground, and making a measurement by activating a sensor only for a period while the user's foot is in contact with the ground. However, in the method of PTL 2, since a high-speed operation is required to measure the moments of landing and leaving during walking, if the data acquisition interval is prolonged, a disadvantage that the landing and leaving cannot be discriminated and power saving cannot be achieved is brought about.

According to the system of PTL 2, there is a possibility that power consumption can be reduced by using an acceleration sensor to control whether data transmission is permitted and cut power of a transmitter. However, the system of PTL 2 has a disadvantage that effective power saving cannot be achieved because arithmetic calculations continue even in a situation where data transmission is not performed. Since the system of PTL 2 does not have an erroneous detection prevention measure, there is a disadvantage that a large amount of power is consumed because of erroneous transmission due to erroneous detection and is unsuitable for power saving in walking measurement.

An object of the present invention is to provide a measurement device capable of saving power in a sensor that acquires gait data of a user in order to solve the above disadvantages.

### Solution to Problem

A measurement device according to one aspect of the present invention includes: a data acquisition unit that measures a sensor detection value by a sensor in at least two operation modes including a first mode with low power and a second mode for operating at a high speed, transmits a trigger signal in response to the sensor detection value exceeding a first threshold value while operating in the first mode, transmits a first notification signal notifying that a user has started walking in response to the sensor detection value having exceeded a second threshold value a prescribed number or more of times during a prescribed period of time while operating in the second mode, and starts measuring gait data including a walking characteristic of the user wearing the sensor based on the sensor detection value; and a control unit that switches the operation mode of the data acquisition unit to the second mode in response to receiving the trigger signal, and switches the operation mode of the data acquisition unit to the first mode in response to a predetermined condition being satisfied after reception of the first notification signal.

A measurement method according to one aspect of the present invention is implemented by a measurement device that measures a sensor detection value in at least two operation modes including a first mode with low power and a second mode for operating at a high speed, the control method including: outputting a trigger signal in response to the sensor detection value exceeding a first threshold value while operating in the first mode; switching the operation mode to the second mode in response to the trigger signal; generating a first notification signal notifying that a user wearing the sensor has started walking, in response to the sensor detection value having exceeded a second threshold value a prescribed number or more of times during a prescribed period of time while operating in the second mode; starting measuring gait data including a walking characteristic of the user based on the sensor detection value; and switching the operation mode to the first mode in response to a predetermined condition being satisfied after generation of the first notification signal.

A program according to one aspect of the present invention is a program for operating a measurement device that measures a sensor detection value in at least two operation modes including a first mode with low power and a second mode for operating at a high speed, the program causing a computer to execute: a process of outputting a trigger signal in response to the sensor detection value exceeding a first threshold value while operating in the first mode; a process of switching the operation mode to the second mode in response to the trigger signal; a process of generating a first notification signal notifying that a user wearing the sensor has started walking, in response to the sensor detection value having exceeded a second threshold value a prescribed number or more of times during a prescribed period of time while operating in the second mode; a process of starting measuring gait data including a walking characteristic of the user based on the sensor detection value; and a process of switching the operation mode to the first mode in response to a predetermined condition being satisfied after generation of the first notification signal.

### Advantageous Effects of Invention

According to the present invention, it is possible to provide a measurement device capable of saving power in a sensor that acquires gait data of a user.

### BRIEF DESCRIPTION OF DRAWINGS

FIG. 1 is a block diagram for explaining an example of the configuration of a measurement device according to a first example embodiment of the present invention.

FIG. 2 is a conceptual diagram for explaining a human walking cycle.

FIG. 3 is a block diagram for explaining an example of the configuration of a data acquisition unit of the measurement device according to the first example embodiment of the present invention.

FIG. 4 is a block diagram for explaining an example of the configuration of a control unit of the measurement device according to the first example embodiment of the present invention.

FIG. 5 is a flowchart for explaining an example of the operation in a low-power mode of the data acquisition unit of the measurement device according to the first example embodiment of the present invention.

FIG. 6 is a flowchart for explaining an example of the operation during mode switching of the control unit of the measurement device according to the first example embodiment of the present invention.

FIG. 7 is a flowchart for explaining an example of the operation in a normal mode of the data acquisition unit of the measurement device according to the first example embodiment of the present invention.

FIG. 8 is a block diagram for explaining an example of the configuration of a measurement device according to the first example embodiment of the present invention.

FIG. 9 is a flowchart for explaining an example of the operation during mode switching of a control unit of the measurement device according to the first example embodiment of the present invention.

FIG. 10 is a flowchart for explaining an example of the operation in a normal mode of a data acquisition unit of the measurement device according to the first example embodiment of the present invention.

FIG. 11 is a block diagram for explaining an example of the configuration of a measurement device according to a third example embodiment of the present invention.

FIG. 12 is a flowchart for explaining an example of the operation in a low-power mode of a data acquisition unit of the measurement device according to the third example embodiment of the present invention.

FIG. 13 is a flowchart for explaining an example of the operation in a normal mode of the data acquisition unit of the measurement device according to the third example embodiment of the present invention.

FIG. 14 depicts acceleration waveforms in a perpendicular direction (positive in the upward direction) relating to walking and ankle rotational motions measured in the low-power mode in an example of the present invention.

FIG. 15 is a histogram of the acceleration waveforms in the perpendicular direction (positive in the upward direction) relating to walking and ankle rotational motions measured in the low-power mode in the example of the present invention.

FIG. 16 depicts acceleration waveforms acquired when a measurement device according to the example of the present invention worn on a test subject.

FIG. 17 depicts acceleration waveforms in a horizontal direction (positive in a forward direction) during walking and ankle rotational motions measured in the normal mode in the example of the present invention.

FIG. 18 is a block diagram for explaining an example of a hardware configuration for achieving the measurement device according to each example embodiment of the present invention.

### EXAMPLE EMBODIMENT

Modes for carrying out the present invention will be described below with reference to the accompanying drawings. However, while the example embodiments described below are limited to technologically preferred ones for carrying out the present invention, the scope of the invention is not limited to the following. In all the figures used in the following explanation of the example embodiments, the same reference signs are given to similar portions unless there is a particular reason. In the following example embodiments, a repetitive description of similar configuration and operation is omitted in some cases.

### First Example Embodiment

First, a measurement device according to a first example embodiment of the present invention will be described with reference to the drawings. The measurement device of the present example embodiment achieves power saving for a sensor worn on a foot part to acquire gait data of a user. Gait represents a manner in which humans and animals walk. Gait includes the step length (left or right, for one step), step length (for two steps), rhythm, speed, mechanical basis, direction of travel, foot angle, waist angle, ability to crouch, and the like.

(Configuration)

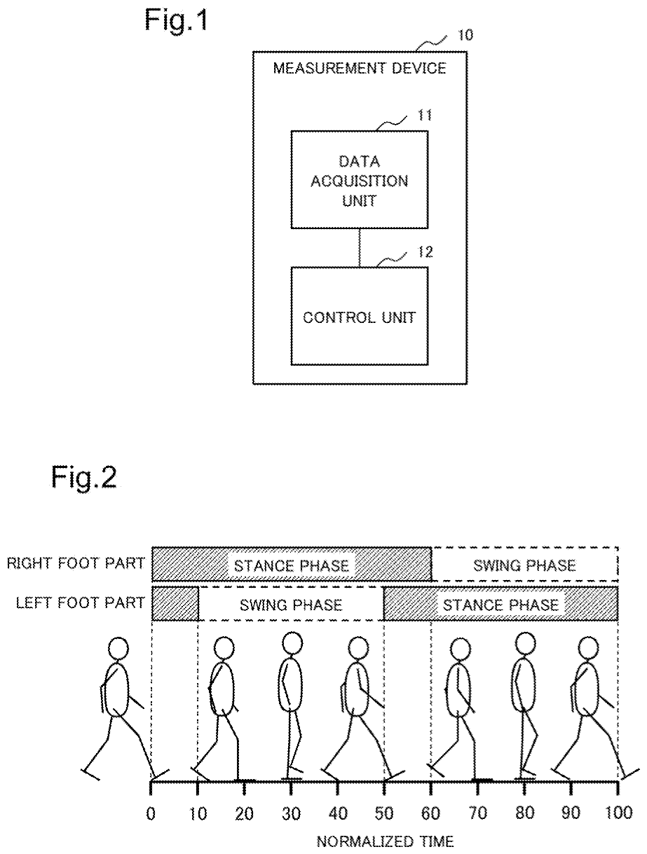

FIG. 1 is a block diagram illustrating an example of the configuration of a measurement device 10 of the present example embodiment. As illustrated in FIG. 1, the measurement device 10 includes a data acquisition unit 11 and a control unit 12.

The data acquisition unit 11 is worn on a foot part of a user. The data acquisition unit 11 acquires gait data of the user. For example, the data acquisition unit 11 is achieved by an inertial measurement unit (IMU). Hereinafter, it is assumed that the data acquisition unit 11 measures acceleration and angular velocity as gait data. The objects to be measured by the data acquisition unit 11 are not limited to acceleration and angular velocity.

The data acquisition unit 11 operates in at least two operation modes including a low-power mode (also referred to as a first mode) and a normal mode (also referred to as a second mode) under the control of the control unit 12. For example, while operating in the low-power mode, the data acquisition unit 11 measures the acceleration in an ultra-low-speed mode and stops the measurement of the angular velocity. For example, while operating in the normal mode, the data acquisition unit 11 measures physical quantities such as acceleration and angular velocity in a high-speed mode.

When the physical quantity under measurement exceeds a first threshold value, the data acquisition unit 11 operating in the low-power mode outputs a trigger signal to the control unit 12. The data acquisition unit 11 switches the operation mode to the normal mode when receiving a mode switching signal according to the output trigger signal from the control unit 12.

For example, when the data acquisition unit 11 includes an acceleration sensor, the acceleration sensor detects the acceleration of a foot part when the foot of the user with the measurement device 10 worn moves. The data acquisition unit 11 discriminates whether the detected acceleration has exceeded the first threshold value set beforehand, and determines that walking has been started when the detected acceleration has exceeded the first threshold value. When the acceleration has exceeded the first threshold value, the trigger signal is output from the data acquisition unit 11 to the control unit 12, and the control unit 12 being dormant is activated.

When the operation mode is switched from the low-power mode to the normal mode, the data acquisition unit 11 verifies whether the gait data has exceeded a second threshold value. For example, the data acquisition unit 11 verifies whether the physical quantity under measurement has exceeded the second threshold value.

When the physical quantity under measurement has exceeded the second threshold value, the data acquisition unit 11 starts counting with a counter. At this time, the data acquisition unit 11 counts the number of times that the physical quantity under measurement has exceeded the second threshold value within a prescribed period of time such as S seconds (S denotes a positive real number). When the physical quantity under measurement has exceeded the second threshold value a number of times greater than a predetermined count number (N times) within the prescribed period of time, the data acquisition unit 11 outputs a notification signal (also referred to as a first notification signal) notifying the start of measurement to the control unit 12, and starts measuring the gait data (N denotes a natural number). Then, the data acquisition unit 11 transmits the measured gait data to a host system or an external system or the like.

On the other hand, when the physical quantity under measurement has not exceeded the second threshold value a number of times greater than the predetermined count number (N times) within the prescribed period of time, the data acquisition unit 11 continues the measurement of the physical quantity in the normal mode. At this time, the data acquisition unit 11 does not start the measurement of the gait data. The data acquisition unit 11 may be configured to transmit a notification signal requesting the switching of the operation mode, to the control unit 12 when the physical quantity under measurement has not exceeded the second threshold value a number of times greater than the predetermined count number (N times) within the prescribed period of time.

The data acquisition unit 11 operating in the normal mode stops the measurement of the gait data and switches the operation mode to the low-power mode when receiving the mode switching signal from the control unit 12.

The data acquisition unit 11 is only required to monitor the acceleration and the angular velocity under measurement and discriminate whether any of the acceleration and the angular velocity has exceeded the second threshold value within the prescribed period of time. Setting as to which of the acceleration and the angular velocity is employed as the physical quantity handled for the determination, and on which axis among the x-axis, y-axis, and z-axis the discrimination is to be made is allowed without restriction. The physical quantities handled for the determination may be freely combined. The data acquisition unit 11 regards that walking has been started if the physical quantity to be determined has exceeded the second threshold value the prescribed number of times (N times) within the prescribed period of time (S seconds), and the data acquisition unit 11 is caused to operate in the normal mode.

The control unit 12 is connected to the data acquisition unit 11. The control unit 12 controls the operation of the data acquisition unit 11 according to the value of the physical quantity measured by the data acquisition unit 11. The control unit 12 maintains the dormant state until receiving the trigger signal from the data acquisition unit 11. The control unit 12 can be achieved by a microcomputer.

When receiving the trigger signal from the data acquisition unit 11, the control unit 12 in the dormant state activates its own device if a predetermined waiting period of time has elapsed since the last mode switching. On the other hand, if the predetermined period of waiting time has not elapsed since the last mode switching, the control unit 12 maintains the dormant state.

The control unit 12 that has been activated in response to the trigger signal from the data acquisition unit 11 transmits the mode switching signal for switching the operation mode of the data acquisition unit 11 to the normal mode, to the data acquisition unit 11.

The control unit 12 also receives the notification signal for the start of measurement or malfunction from the data acquisition unit 11 that has been switched to the normal mode.

When the notification signal received from the data acquisition unit 11 is a notification of the start of measurement, the control unit 12 starts counting the elapsed time since the start of measurement. When a predetermined period of time (M seconds) has elapsed, the control unit 12 transmits the mode switching signal for switching the operation mode of the data acquisition unit 11 to the low-power mode, to the data acquisition unit 11 (M denotes a positive real number).

On the other hand, when the notification signal received from the data acquisition unit 11 is a notification of malfunction, the control unit 12 transmits the mode switching signal for switching the operation mode of the data acquisition unit 11 to the low-power mode, to the data acquisition unit 11.

After transmitting the mode switching signal for switching the operation mode of the data acquisition unit 11 to the low-power mode to the data acquisition unit 11, the control unit 12 shifts to the dormant state for a predetermined period (K hours).

The above is the description of an example of the configuration of the measurement device 10. The measurement device 10 in FIG. 1 is an example, and the configuration of the measurement device 10 of the present example embodiment is not limited to the original form.

[Walking Cycle]

A human walking cycle will now be described with reference to the drawings. FIG. 2 is a conceptual diagram for explaining a human walking cycle with the right foot as a reference. The lateral axis illustrated below a pedestrian in FIG. 2 denotes a normalized time obtained by normalizing a time course related to walking of a human. In the following description, attention will be paid to the right foot, but the same applies to the left foot.

The human walking cycle is roughly divided into a stance phase and a swing phase. The stance phase of the right foot is a period from a state in which the heel of the right foot is in contact with the ground to a state in which the bottom surface of the left foot makes complete contact with the ground and the toe of the right foot leaves the ground. The stance phase accounts for 60% of the entire walking cycle. The swing phase of the right foot is a period from a state in which the bottom surface of the left foot makes complete contact with the ground and the toe of the right foot leaves the ground to a state in which the heel of the right foot makes contact with the ground again. The swing phase accounts for 40% of the entire walking cycle.

A rotational motion of the ankle joint in the vertical direction after the heel of the right foot makes contact with the ground generates an impact when the entire sole contacts the ground. Pressure exerted on the ground by the toe of the right foot is generated when a forward posture is formed by the left foot whose heel makes contact with the ground and the right foot whose toe leaves the ground, which appears during a period between the latter term of the stance phase and the early term of the swing phase of the right foot, and in order to overcome the frictional force with the ground, horizontal acceleration is generated by the force of muscles exerted on the forward movement by a person.

In particular, the impact generated when the sole makes complete contact with the ground is due to the body weight, and the acceleration caused by the rotational motion of the ankle without walking, which is a factor of malfunction, is the force exerted by muscles. In an ordinary human, the acceleration exerted by the force of muscles is no match for the impact force due to the body weight. Accordingly, if a threshold value is appropriately set by utilizing this difference, walking can be detected.

[Data Acquisition Unit]

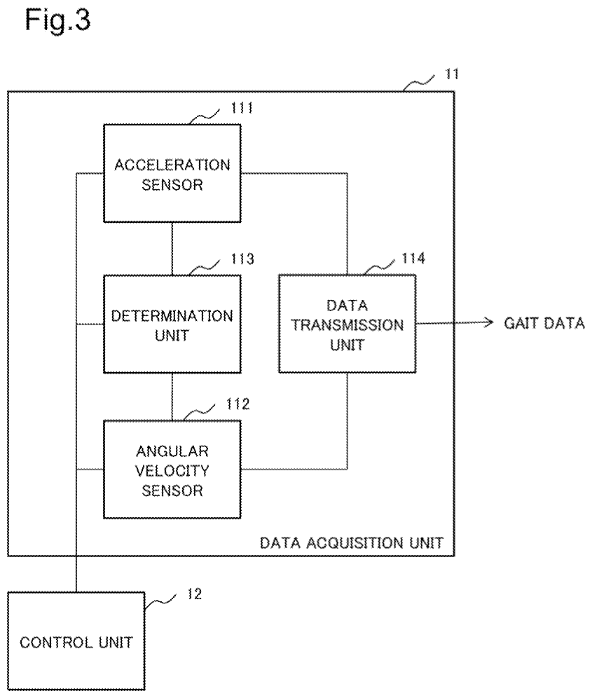

Next, an example of the detailed configuration of the data acquisition unit 11 will be described with reference to the drawings. FIG. 3 is a block diagram illustrating an example of the detailed configuration of the data acquisition unit 11. As illustrated in FIG. 3, the data acquisition unit 11 includes an acceleration sensor 111, an angular velocity sensor 112, a determination unit 113, and a data transmission unit 114.

The acceleration sensor 111 is a sensor that measures acceleration. For example, a sensor that detects acceleration by any technique including a piezoelectric type, a piezoresistance type, a capacitance type, and the like may be applied as the acceleration sensor 111. The acceleration sensor 111 operates in at least two operation modes including an ultra-low-power mode with a low sampling rate and a high-speed mode for operating at a high speed. The operation mode of the acceleration sensor 111 is switched under the control of the control unit 12.

The angular velocity sensor 112 is a sensor that measures angular velocity. For example, a sensor that measures angular velocity by any technique including a vibration type, a capacitance type, and the like can be applied as the angular velocity sensor 112. The angular velocity sensor 112 operates in at least one operation mode including a high-speed mode. The operation mode of the angular velocity sensor 112 is switched under the control of the control unit 12.

The low-power mode is an operation mode in which the acceleration sensor 111 is in the ultra-low-power mode, and the angular velocity sensor 112 is in a stopped state. Meanwhile, the normal mode is an operation mode in which the acceleration sensor 111 is in the high-speed mode, and the angular velocity sensor 112 is also in the high-speed mode.

The determination unit 113 determines whether the acceleration measured by the acceleration sensor 111 has exceeded the first threshold value. When the acceleration measured by the acceleration sensor 111 has exceeded the first threshold value, the determination unit 113 outputs the trigger signal to the control unit 12.

When the operation mode of the data acquisition unit 11 is switched to the normal mode in response to the switching of the operation mode by the control unit 12, the determination unit 113 counts the number of times that the acceleration measured by the acceleration sensor 111 has exceeded the second threshold value within the prescribed period of time. When the number of times that the acceleration measured by the acceleration sensor 111 has exceeded the second threshold value within the prescribed period of time is equal to or more than a predetermined count, the determination unit 113 outputs the notification signal notifying the start of measurement, to the control unit 12. Then, the determination unit 113 causes the acceleration sensor 111 and the angular velocity sensor 112 to output data under measurement to the data transmission unit 114.

The data transmission unit 114 transmits the data measured by the acceleration sensor 111 and the angular velocity sensor 112 as the gait data. For example, the data transmission unit 114 transmits the gait data to a host system or an external system or the like. The gait data transmitted from the data transmission unit 114 is mainly used for the purpose of studies of walking of the user.

The above is an example of the description of the detailed configuration of the data acquisition unit 11. The configuration of the data acquisition unit 11 illustrated in FIG. 3 is an example, and the configuration of the data acquisition unit 11 of the present example embodiment is not limited to the original form.

[Control Unit]

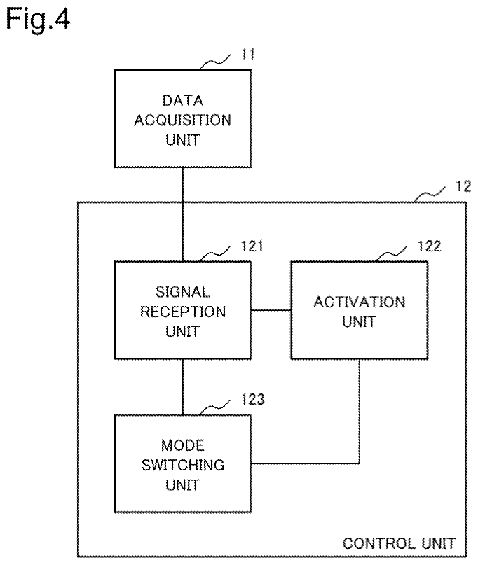

Next, the detailed configuration of the control unit 12 will be described with reference to the drawings. FIG. 4 is a block diagram illustrating an example of the detailed configuration of the control unit 12. As illustrated in FIG. 4, the control unit 12 includes a signal reception unit 121, an activation unit 122, and a mode switching unit 123.

The signal reception unit 121 receives a signal from the data acquisition unit 11. For example, the signal reception unit 121 receives the trigger signal and the notification signal from the data acquisition unit 11. When receiving the trigger signal, the signal reception unit 121 outputs the received trigger signal to the activation unit 122. When receiving the notification signal, the signal reception unit 121 outputs the received notification signal to the mode switching unit 123.

The activation unit 122 receives the trigger signal from the signal reception unit 121. When receiving the trigger signal, the activation unit 122 activates the control unit 12 based on the elapsed time since the last time the mode was switched. The activation unit 122 activates the control unit 12 when the elapsed time since the last time the mode was switched has elapsed by a predetermined elapsed time. On the other hand, when the elapsed time since the last time the mode was switched has not elapsed by the predetermined elapsed time, the dormant state of the control unit 12 is maintained.

When acquiring a signal instructing to shift the control unit 12 to the dormant state from the mode switching unit 123, the activation unit 122 shifts the control unit 12 to the dormant state.

When the control unit 12 is activated, the mode switching unit 123 transmits the mode switching signal for switching the operation mode of the data acquisition unit 11 to the normal mode, to the data acquisition unit 11.

When receiving the notification signal from the signal reception unit 121, the mode switching unit 123 performs a process according to the notification content. When receiving the notification signal notifying the start of measurement, the mode switching unit 123 performs counting only for a predetermined period. The mode switching unit 123 transmits the mode switching signal for switching the operation mode of the data acquisition unit 11 to the low-power mode, to the data acquisition unit 11 when the predetermined period has elapsed.

When transmitting the mode switching signal for switching the operation mode to the low-power mode to the data acquisition unit 11, the mode switching unit 123 outputs a signal instructing to shift the control unit 12 to the dormant state, to the activation unit 122.

The above is the description of an example of the detailed configuration of the control unit 12. The configuration of the control unit 12 illustrated in FIG. 4 is an example, and the configuration of the control unit 12 of the present example embodiment is not limited to the original form.

(Operation)

Next, the operation of the measurement device 10 of the present example embodiment will be described with reference to the drawings. In the following, the operation of the data acquisition unit 11 in each operation mode and the operation of the control unit will be described with reference to separate flowcharts.

[Low-Power Mode]

First, an example of the operation of the data acquisition unit 11 in the low-power mode will be described with reference to the drawings. In the low-power mode, the angular velocity sensor is set to a stopped state and the acceleration sensor is set to operate in the ultra-low-speed mode.

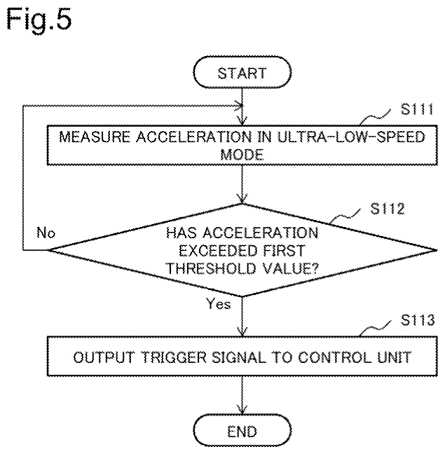

FIG. 5 is a flowchart for explaining the operation of the data acquisition unit 11 operating in the low-power mode. In the process in line with the flowchart in FIG. 5, the data acquisition unit 11 will be described as the subject of the operation.

In FIG. 5, first, the data acquisition unit 11 measures acceleration in the ultra-low-speed mode (step S111).

Here, the data acquisition unit 11 verifies whether the measured acceleration has exceeded the first threshold value (step S112).

When the measured acceleration has exceeded the first threshold value (Yes in step S112), the data acquisition unit 11 transmits the trigger signal to the control unit 12 (step S113). On the other hand, when the measured acceleration is equal to or less than the first threshold value (No in step S112), the process returns to step S111.

The above is the description of an example of the operation of the data acquisition unit 11 in the low-power mode. The operation of the data acquisition unit 11 illustrated in FIG. 5 is an example, and the operation of the data acquisition unit 11 in the low-power mode is not limited to the original method.

[Mode Switching]

Next, an example of the operation of the control unit 12 will be described with reference to the drawings. Here, the operation after the control unit 12 set in the dormant state receives the trigger signal is concerned.

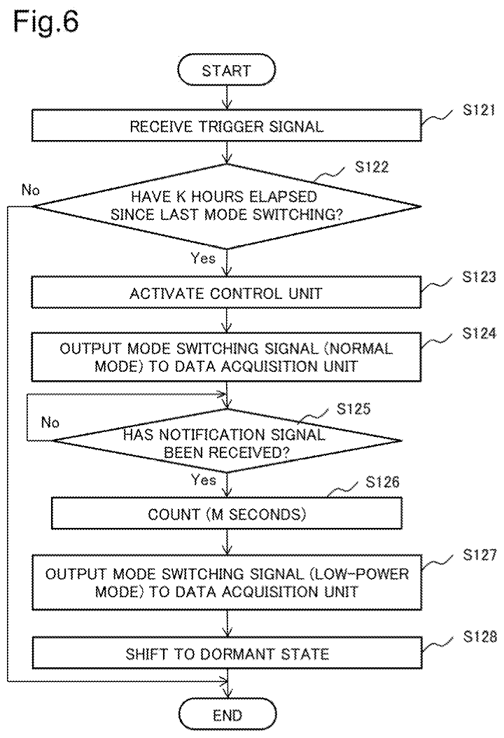

FIG. 6 is a flowchart for explaining the operation of the control unit 12. In the process in line with the flowchart in FIG. 6, the control unit 12 will be described as the subject of the operation.

First, in FIG. 6, the control unit 12 receives the trigger signal from the data acquisition unit 11 (step S121).

Here, the control unit 12 confirms the elapsed time since the last switching of the operation mode (step S122). When the predetermined period (K hours) has elapsed since the last switching of the operation mode (Yes in step S122), the control unit 12 activates the own control unit 12 (the same control unit 12) (step S123). On the other hand, when the predetermined period (K hours) has not elapsed since the last switching of the operation mode (No in step S122), the control unit 12 continues the dormant state. Step S122 may be omitted, and the control unit 12 may activate the own control unit 12 (step S123) at the stage of receiving the trigger signal (step S121).

After activating the own control unit 12 in step S123, the control unit 12 outputs the mode switching signal for switching the operation mode of the data acquisition unit 11 to the normal mode from the low-power mode, to the data acquisition unit 11 (step S124).

Here, the control unit 12 waits to receive the notification signal from the data acquisition unit 11 (step S125). When the notification signal is not received from the data acquisition unit 11 (No in step S125), the control unit 12 waits for the reception of the notification signal. On the other hand, when the notification signal is received from the data acquisition unit 11 (Yes in step S125), counting the predetermined period of time (M seconds) is started (step S126).

Next, when the predetermined period of time (M seconds) has elapsed while counting, the control unit 12 outputs the mode switching signal for switching the operation mode of the data acquisition unit 11 to the low-power mode from the normal mode, to the data acquisition unit 11 (step S127).

Then, the control unit 12 shifts to the dormant state (step S128).

The above is the description of an example of the operation of the control unit 12. The operation of the control unit 12 illustrated in FIG. 6 is an example, and the operation of the control unit 12 is not limited to the original method.

[Normal Mode]

Next, an example of the operation of the data acquisition unit 11 in the normal mode will be described with reference to the drawings. In the normal mode, both of the acceleration sensor and the angular velocity sensor are set to operate in the high-speed mode.

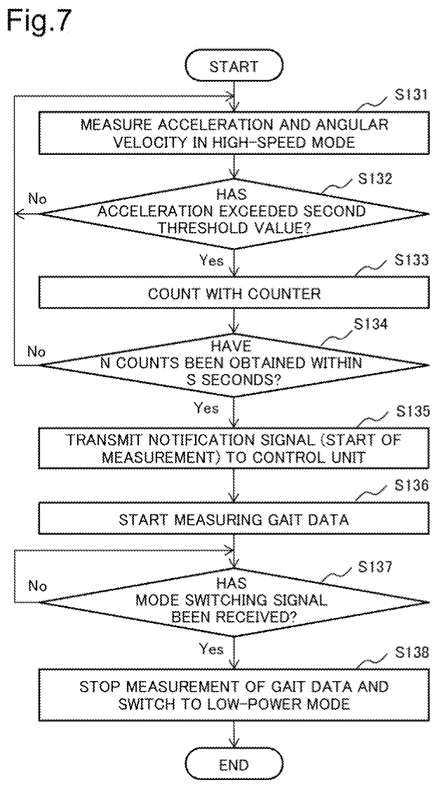

FIG. 7 is a flowchart for explaining the operation of the data acquisition unit 11 operating in the normal mode. In the process in line with the flowchart in FIG. 7, the data acquisition unit 11 will be described as the subject of the operation. In the process in line with the flowchart in FIG. 7, a case where acceleration is employed as the physical quantity to be determined using the threshold value will be described.

In FIG. 7, first, the data acquisition unit 11 measures acceleration and angular velocity in the high-speed mode (step S131).

Here, the data acquisition unit 11 determines whether the acceleration under measurement has exceeded the second threshold value (step S132). When the acceleration under measurement has exceeded the second threshold value (Yes in step S132), the data acquisition unit 11 counts the number of times that the acceleration has exceeded the second threshold value, with a counter (step S133). On the other hand, when the acceleration under measurement has not exceeded the second threshold value (No in step S132), the process returns to step S131. When the acceleration under measurement has not exceeded the second threshold value within the preset period of time, the process may proceed to step S137.

When the acceleration has exceeded the second threshold value the prescribed number (N counts) or more of times within the prescribed period of time (S seconds) (Yes in step S134), the data acquisition unit 11 transmits the notification signal notifying the start of measurement, to the control unit 12 (step S135). On the other hand, when the acceleration has not exceeded the second threshold value the prescribed number (N counts) or more of times within the prescribed period of time (S seconds) (No in step S134), the process returns to step S131.

After step S135, the data acquisition unit 11 starts measuring the gait data (step S136). The data acquisition unit 11 continues the measurement of the gait data until receiving the mode switching signal.

After step S136, when receiving the mode switching signal for switching the operation mode to the low-power mode from the normal mode (Yes in step S137), the data acquisition unit 11 stops the measurement of the gait data and switches the operation mode to the low-power mode (step S138). On the other hand, when the mode switching signal is not received (No in step S137), the data acquisition unit 11 continues the measurement of the gait data.

The above is the description of an example of the operation of the data acquisition unit 11 in the normal mode. The operation of the data acquisition unit 11 illustrated in FIG. 7 is an example, and the operation of the data acquisition unit 11 in the normal mode is not limited to the original method.

As described above, the measurement device of the present example embodiment includes the data acquisition unit and the control unit. The data acquisition unit measures a sensor detection value in at least two operation modes including the first mode with low power and the second mode for operating at a high speed.

The measurement device transmits the trigger signal when the value detected by the sensor exceeds the first threshold value while operating in the first mode. The measurement device transmits the first notification signal notifying that a user wearing the sensor has started walking, when the value detected by the sensor has exceeded the second threshold value a prescribed number or more of times during a prescribed period of time while operating in the second mode. Then, the measurement device starts measuring the gait data of the user including a walking characteristic based on the value detected by the sensor. The control unit switches the operation mode of the data acquisition unit to the second mode when receiving the trigger signal. The control unit switches the operation mode of the data acquisition unit to the first mode when a predetermined condition is satisfied after receiving the first notification signal.

For example, the data acquisition unit includes an acceleration sensor that detects acceleration and an angular velocity sensor that detects angular velocity. The control unit operates either one of the acceleration sensor and the angular velocity sensor with low power consumption and stops the operation of the other of the acceleration sensor and the angular velocity sensor in the first mode, and operates both of the acceleration sensor and the angular velocity sensor at a high speed in the second mode.

For example, the control unit is activated when receiving the trigger signal to switch the operation mode of the data acquisition unit to the second mode, and when the predetermined condition is satisfied after receiving the first notification signal, the control unit switches the operation mode of the data acquisition unit to the first mode to shift to a dormant state. For example, the control unit does not activate the own control unit when receiving the trigger signal, in a case where a predetermined period has not elapsed since the last switching of the operation mode of the data acquisition unit.

For example, the control unit switches the operation mode of the data acquisition unit to the first mode from the second mode at a stage when a predetermined period of time has elapsed since the first notification signal was received.

According to the present example embodiment, by efficiently switching between the first mode with low power and the second mode for operating at a high speed at an appropriate timing, power saving and long life of the sensor for measuring gait data can be achieved.

The measurement device of the present example embodiment can be configured in such a way that the working period in the normal mode is limited, the measurement is regarded as being successful when having worked only for a preset predetermined period of time, a shift to the low-power mode is made to put the control unit into the dormant state, and the trigger signal is not accepted during the dormant period. With such a configuration, the working period of the measurement device can be set to the minimum required, and accordingly further power saving for the measurement device that acquires the gait data of the user is enabled.

### Second Example Embodiment

Next, a measurement device according to a second example embodiment of the present invention will be described with reference to the drawings. The measurement device of the present example embodiment is different from the measurement device of the first example embodiment in that it is determined that an erroneous operation occurs when the acceleration under measurement has not exceeded the second threshold value a predetermined number of times within a predetermined period of time.

(Configuration)

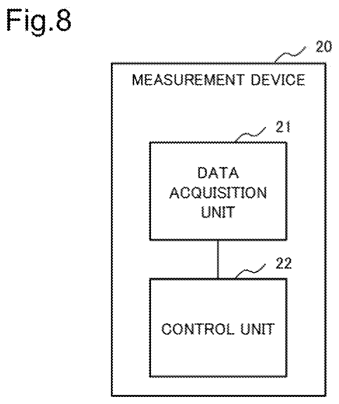

FIG. 8 is a block diagram illustrating an example of the configuration of a measurement device 20 of the present example embodiment. As illustrated in FIG. 8, the measurement device 20 includes a data acquisition unit 21 and a control unit 22. Since each of the data acquisition unit 21 and the control unit 22 of the present example embodiment has a configuration similar to the configuration of each of the data acquisition unit 11 and the control unit 12 of the first example embodiment, a detailed description will be omitted and differences will be described.

When the operation mode is switched to the normal mode in response to the switching of the operation mode by the control unit 22, the data acquisition unit 21 counts the number of times that the acceleration measured by an acceleration sensor has exceeded the second threshold value within the prescribed period of time. When the number of times that the acceleration measured by the acceleration sensor has exceeded the second threshold value within the prescribed period of time is equal to or more than a prescribed number of times, the data acquisition unit 21 outputs a notification signal (also referred to as a first notification signal) notifying the start of measurement, to the control unit 22. Then, the data acquisition unit 21 transmits data under measurement from the acceleration sensor and an angular velocity sensor. On the other hand, when the number of times that the acceleration measured by the acceleration sensor has exceeded the second threshold value within the prescribed period of time falls below the predetermined number of times, the data acquisition unit 21 outputs a notification signal (also referred to as a second notification signal) notifying the malfunction, to the control unit 22.

When receiving the notification signal from the data acquisition unit 21, the control unit 22 performs a process according to the notification content. When receiving the notification signal (first notification signal) notifying the start of measurement, the control unit 22 performs counting only for the predetermined period of time. The control unit 22 transmits the mode switching signal for switching the operation mode of the data acquisition unit 21 to the low-power mode, to the data acquisition unit 21 when the predetermined period of time has elapsed. On the other hand, when receiving the notification signal (second notification signal) notifying the malfunction, the control unit 22 does not perform counting but transmits the mode switching signal for switching the operation mode of the data acquisition unit 21 to the low-power mode, to the data acquisition unit 21.

The above is the description of a difference in the configuration of the measurement device 20 of the present example embodiment from the configuration of the measurement device 10 of the first example embodiment.

Next, an example of the operation of the measurement device 20 of the present example embodiment will be described with reference to the drawings. Since the operation of the data acquisition unit 21 in the low-power mode is similar to the operation of the first example embodiment, a detailed description thereof will be omitted.

[Mode Switching]

Here, the operation after the control unit 22 set in the dormant state receives the trigger signal will be described.

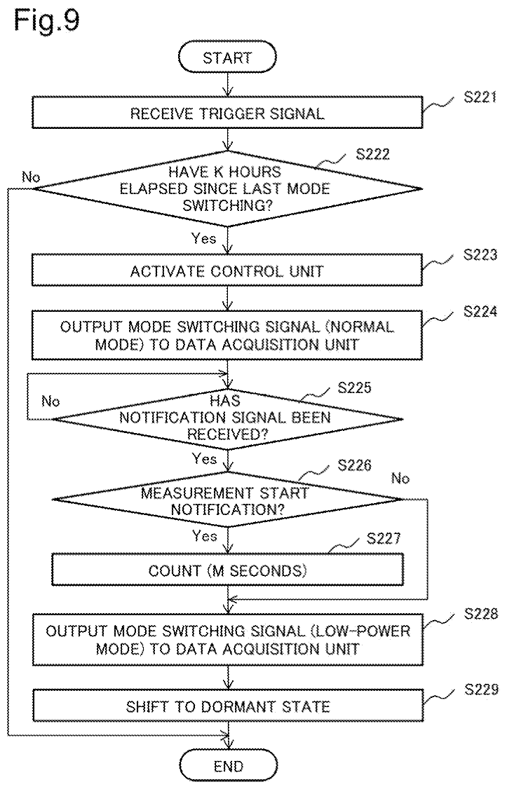

FIG. 9 is a flowchart for explaining the operation of the control unit 22. In the process in line with the flowchart in FIG. 9, the control unit 22 will be described as the subject of the operation.

First, in FIG. 9, the control unit 22 receives the trigger signal from the data acquisition unit 21 (step S221).

Here, the control unit 22 confirms the elapsed time since the last switching of the operation mode (step S222). When the predetermined period (K hours) has elapsed since the last switching of the operation mode (Yes in step S222), the control unit 22 activates the own control unit 22 (step S223). On the other hand, when the predetermined period (K hours) has not elapsed since the last switching of the operation mode (No in step S222), the control unit 12 continues the dormant state. Step S222 may be omitted, and the control unit 22 may activate the own control unit 22 (step S223) at the stage of receiving the trigger signal (step S221).

After activating the own control unit 22 in step S223, the control unit 22 outputs the mode switching signal for switching the operation mode of the data acquisition unit 21 to the normal mode from the low-power mode, to the data acquisition unit 21 (step S224).

Here, the control unit 22 waits to receive the notification signal from the data acquisition unit 21 (step S225). When the notification signal is not received from the data acquisition unit 21 (No in step S225), the control unit 22 waits for the reception of the notification signal. On the other hand, when the notification signal is received from the data acquisition unit 21 (Yes in step S225), the control unit 22 interprets the content of the received notification signal (step S226).

When interpreting the notification signal as a measurement start notification (Yes in step S226), the control unit 22 starts counting the predetermined period of time (M seconds) (step S227). On the other hand, when the control unit 22 interprets the notification signal as an erroneous operation notification instead of the measurement start notification (No in step S226), the process proceeds to step S228.

Next, when the predetermined period of time (M seconds) has elapsed while counting, the control unit 22 outputs the mode switching signal for switching the operation mode of the data acquisition unit 21 to the low-power mode from the normal mode, to the data acquisition unit 21 (step S228).

Then, the control unit 22 shifts to the dormant state (step S229).

The above is the description of an example of the operation of the control unit 22. The operation of the control unit 22 illustrated in FIG. 9 is an example, and the operation of the control unit 22 is not limited to the original method.

[Normal Mode]

Next, an example of the operation of the data acquisition unit 21 in the normal mode will be described with reference to the drawings. In the normal mode, both of the acceleration sensor and the angular velocity sensor are set to operate in the high-speed mode.

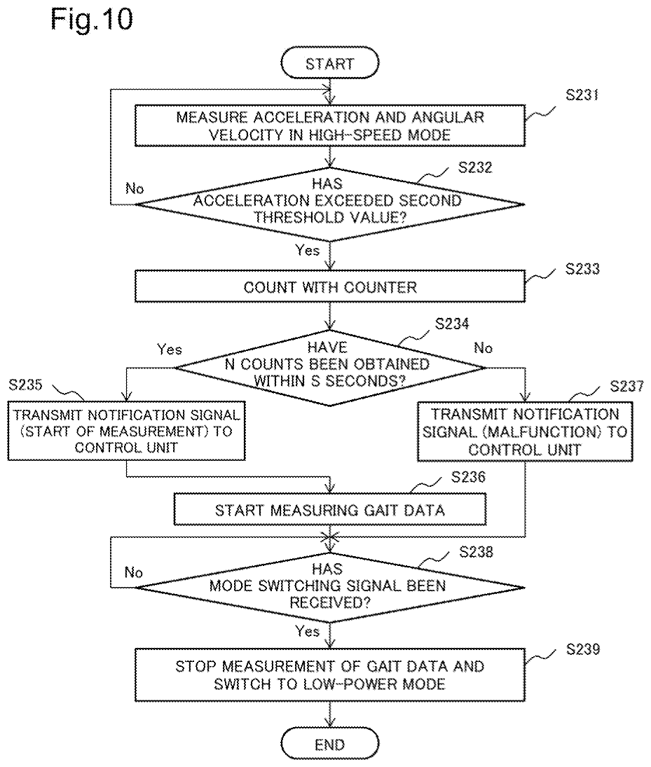

FIG. 10 is a flowchart for explaining the operation of the data acquisition unit 21 operating in the normal mode. In the process in line with the flowchart in FIG. 10, the data acquisition unit 21 will be described as the subject of the operation. In the process in line with the flowchart in FIG. 10, a case where acceleration is employed as the physical quantity to be determined will be described.

In FIG. 10, first, the data acquisition unit 21 measures acceleration and angular velocity in the high-speed mode (step S231).

Here, the data acquisition unit 21 determines whether the acceleration under measurement has exceeded the second threshold value (step S232). When the acceleration under measurement has exceeded the second threshold value (Yes in step S232), the data acquisition unit 21 counts the number of times that the acceleration has exceeded the second threshold value, with a counter (step S233). On the other hand, when the acceleration under measurement has not exceeded the second threshold value (No in step S232), the process returns to step S231. When the acceleration under measurement has not exceeded the second threshold value within the preset period of time, it may be determined that a malfunction has occurred and the process may proceed to step S236.

When the acceleration has exceeded the second threshold value the prescribed number (N counts) or more of times within the prescribed period of time (S seconds) (Yes in step S234), the data acquisition unit 21 transmits the notification signal notifying the start of measurement, to the control unit 12 (step S235).

Then, the data acquisition unit 21 starts measuring the gait data (step S236). The data acquisition unit 21 continues the measurement of the gait data until receiving the mode switching signal.

On the other hand, when the acceleration has not exceeded the second threshold value the prescribed number (N counts) or more of times within the prescribed period of time (S seconds) (No in step S234), the data acquisition unit 21 transmits the notification signal notifying the malfunction, to the control unit 22 (step S237).

After step S236, when receiving the mode switching signal for switching the operation mode to the low-power mode from the normal mode (Yes in step S238), the data acquisition unit 21 stops the measurement of the gait data and switches to the low-power mode (step S239). On the other hand, when the mode switching signal is not received (No in step S238), the data acquisition unit 21 continues the measurement of the gait data.

The above is the description of an example of the operation of the data acquisition unit 21 in the normal mode. The operation of the data acquisition unit 21 illustrated in FIG. 10 is an example, and the operation of the data acquisition unit 21 is not limited to the original method.

As described above, when the value detected by the sensor has not exceeded the second threshold value the prescribed number or more of times during the prescribed period of time while operating in the second mode, the data acquisition unit of the present example embodiment transmits the second notification signal notifying that a malfunction has occurred, to the control unit. Then, when receiving the second notification signal, the control unit of the present example embodiment switches the operation mode of the data acquisition unit to the first mode from the second mode. That is, if the detection value has not exceeded the second threshold value the prescribed number or more of times within the prescribed period of time, the measurement device of the present example embodiment regards that a malfunction has occurred, to set the data acquisition unit to the low-power mode and return the control unit to the dormant state, and shifts to the next discrimination cycle. According to the present example embodiment, a malfunction of the measurement device can be prevented by utilizing the inherent characteristics of the walking waveforms in the first mode for operating at a low sampling rate and the second mode for operating at a high sampling rate.

### Third Example Embodiment

Next, a measurement device according to a third example embodiment of the present invention will be described with reference to the drawings. The measurement device of the present example embodiment is different from the measurement device of the second example embodiment in that logs of the discrimination of the user's walking and the malfunction are learned, and a threshold value, which is usually set by the manufacturer or the user, is automatically set by artificial intelligence (AI).

(Configuration)

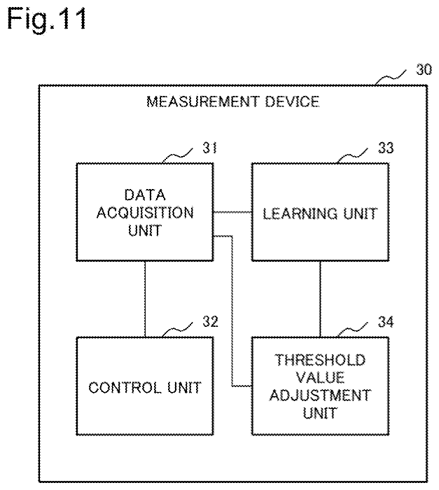

FIG. 11 is a block diagram illustrating an example of the configuration of a measurement device 30 of the present example embodiment. As illustrated in FIG. 11, the measurement device 30 includes a data acquisition unit 31, a control unit 32, a learning unit 33, and a threshold value adjustment unit 34. Since each of the data acquisition unit 31 and the control unit 32 of the present example embodiment has a configuration similar to the configuration of each of the data acquisition unit 21 and the control unit 22 of the second example embodiment, a detailed description will be omitted.

The learning unit 33 records the first threshold value and the second threshold value in a log when it is determined that the physical quantity under measurement has exceeded the first threshold value due to a malfunction. Then, the learning unit 33 inputs the recorded log to a learner, and generates a threshold value adjustment model for adjusting the first threshold value and the second threshold value. For example, the learning unit 33 generates the threshold value adjustment model by inputting the log to a learner having machine learning functions such as supervised learning, unsupervised learning, and reinforcement learning. The learner used by the learning unit 33 is not particularly limited as long as the learner can generate a learning model (threshold value adjustment model) from the first threshold value and the second threshold value recorded as a log.

The threshold value adjustment unit 34 adjusts the first threshold value and the second threshold value for the data acquisition unit 31, using the threshold value adjustment model generated by the learning unit 33. The threshold value adjustment unit 34 feeds back the adjusted first threshold value and second threshold value to the learner.

The above is the description of an example of the configuration of the measurement device 30 of the present example embodiment. The configuration of the measurement device 30 in FIG. 11 is an example, and the configuration of the measurement device 30 of the present example embodiment is not limited.

(Operation)

Next, an example of the operation of the measurement device 30 of the present example embodiment will be described with reference to the drawings. In the following, the operation of the data acquisition unit 31 in each operation mode will be described with reference to separate flowcharts. Since the operation of the control unit 32 is similar to the operation of the control unit 22 of the second example embodiment, the operation of the control unit 32 will be omitted here.

[Low-Power Mode]

First, an example of the operation of the data acquisition unit 31 in the low-power mode will be described with reference to the drawings. In the low-power mode, an angular velocity sensor is set to the dormant state and an acceleration sensor is set to operate in the ultra-low-speed mode.

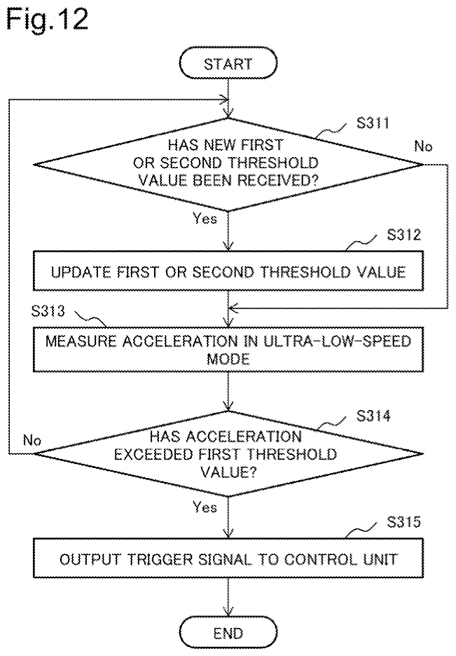

FIG. 12 is a flowchart for explaining the operation of the data acquisition unit 31 operating in the low-power mode. In the process in line with the flowchart in FIG. 12, the data acquisition unit 31 will be described as the subject of the operation.

In FIG. 12, first, when a new first threshold value or second threshold value has been received (Yes in step S311), the data acquisition unit 31 updates the first threshold value or second threshold value with the new threshold value (step S312). On the other hand, when the data acquisition unit 31 has not received a new first threshold value or second threshold value (No in step S311), the process proceeds to step S313.

Next, the data acquisition unit 31 measures the acceleration in the ultra-low-speed mode (step S313).

Here, the data acquisition unit 31 verifies whether the measured acceleration has exceeded the first threshold value (step S314).

When the measured acceleration has exceeded the first threshold value (Yes in step S314), the data acquisition unit 31 transmits the trigger signal to the control unit 12 (step S315). On the other hand, when the measured acceleration is equal to or less than the first threshold value (No in step S314), the process returns to step S311.

The above is the description of an example of the operation of the data acquisition unit 31 in the low-power mode. The operation of the data acquisition unit 31 illustrated in FIG. 12 is an example, and the operation of the data acquisition unit 31 is not limited to the original method.

[Normal Mode]

Next, an example of the operation of the data acquisition unit 31 in the normal mode will be described with reference to the drawings. In the normal mode, both of the acceleration sensor and the angular velocity sensor are set to operate in the high-speed mode.

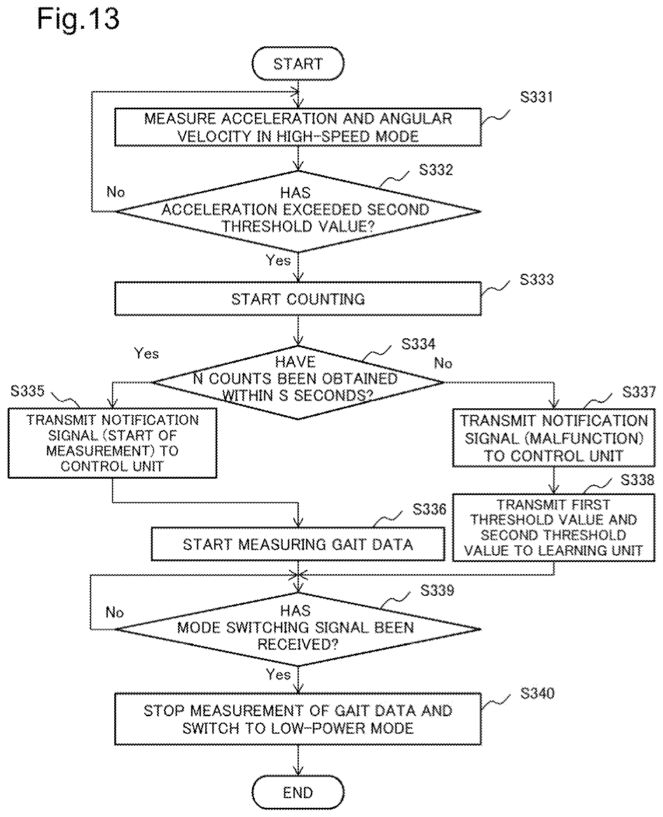

FIG. 13 is a flowchart for explaining the operation of the data acquisition unit 31 operating in the low-power mode. In the process in line with the flowchart in FIG. 13, the data acquisition unit 31 will be described as the subject of the operation. In the process in line with the flowchart in FIG. 13, a case where acceleration is employed as the physical quantity to be determined will be described.

In FIG. 13, first, the data acquisition unit 31 measures acceleration and angular velocity in the high-speed mode (step S331).

Here, the data acquisition unit 11 determines whether the acceleration under measurement has exceeded the second threshold value (step S332). When the acceleration under measurement has exceeded the second threshold value (Yes in step S332), the data acquisition unit 31 counts the number of times that the acceleration has exceeded the second threshold value, with a counter (step S333). On the other hand, when the acceleration under measurement has not exceeded the second threshold value (No in step S332), the process returns to step S331. When the acceleration under measurement has not exceeded the second threshold value within the preset period of time, it may be determined that a malfunction has occurred and the process may proceed to step S336.

When the acceleration has exceeded the second threshold value the prescribed number (N counts) or more of times within the prescribed period of time (S seconds) (Yes in step S334), the data acquisition unit 31 transmits the notification signal notifying the start of measurement, to the control unit 32 (step S335).

Then, the data acquisition unit 31 starts measuring the gait data (step S336). The data acquisition unit 31 continues the measurement of the gait data until receiving the mode switching signal.

On the other hand, when the acceleration has not exceeded the second threshold value the prescribed number (N counts) or more of times within the prescribed period of time (S seconds) (No in step S334), the data acquisition unit 31 transmits the notification signal notifying the malfunction, to the control unit 32 (step S337).

Next, the data acquisition unit 31 transmits the first threshold value and the second threshold value at that time point to the learning unit 33 (step S338).

After step S336, when receiving the mode switching signal for switching the operation mode to the low-power mode from the normal mode (Yes in step S339), the data acquisition unit 31 stops the measurement of the gait data and switches to the low-power mode (step S340). On the other hand, when the mode switching signal is not received (No in step S339), the data acquisition unit 31 continues the measurement of the gait data.

The above is the description of an example of the operation of the data acquisition unit 31 in the normal mode. The operation of the data acquisition unit 31 illustrated in FIG. 13 is an example, and the operation of the data acquisition unit 31 is not limited to the original method.

As described above, the measurement device of the present example embodiment includes the learning unit and the threshold value adjustment unit. The learning unit records the first threshold value and the second threshold value in a log, and inputs the recorded log to the learner to generate a learning model for adjusting the first threshold value and the second threshold value. The threshold value adjustment unit uses the learning model to adjust the first threshold value and the second threshold value used by the data acquisition unit. When the value detected by the sensor has not exceeded the second threshold value the prescribed number or more of times during the prescribed period of time while operating in the second mode, the data acquisition unit of the present example embodiment transmits the second notification signal notifying that a malfunction has occurred, to the control unit. The data acquisition unit transmits the second notification signal to the control unit, and also transmits the first threshold value and the second threshold value at that time point to the learning unit.

For example, the data acquisition unit of the present example embodiment transmits the first notification signal notifying that the user wearing the sensor has started walking, when the value detected by the sensor has fallen below the second threshold value the prescribed number or more of times during the prescribed period of time while operating in the second mode.

That is, the measurement device of the present example embodiment records the first threshold value and the second threshold value when a malfunction was detected, in a log, and inputs the recorded log to the learner together with logs until that time point to generate new first threshold value and second threshold value. Then, the measurement device of the present example embodiment updates the first threshold value and the second threshold value when the malfunction was detected, with the newly generated first threshold value and second threshold value, and continues the measurement thereafter.

According to the measurement device of the present example embodiment, the first threshold value and the second threshold value can be updated according to individual differences and changes in the walking environment. Therefore, according to the measurement device of the present example embodiment, it is possible to flexibly cope with individual differences and changes in the walking environment.

### Example

Here, an example of the measurement device according to the third example embodiment of the present invention will be described with reference to the drawings. In the present example, a simulation was performed to examine the presence or absence of erroneous operation in the low-power mode and the capability in detecting walking in the normal mode, relating to walking and ankle rotational motions when the IMU is worn on an arch part of a foot as the data acquisition unit.

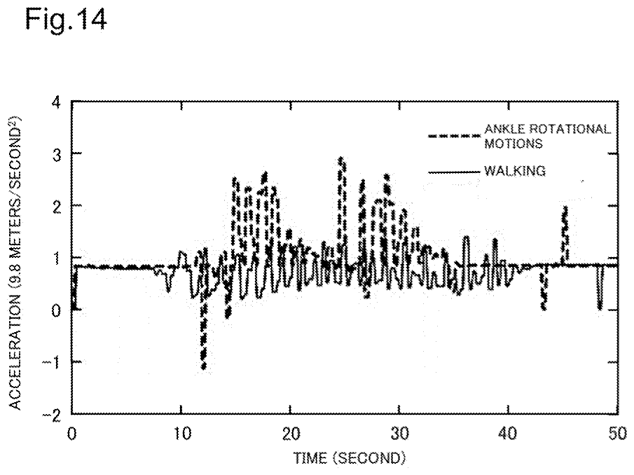

FIG. 14 illustrates acceleration waveforms in the perpendicular direction (positive in the upward direction) relating to walking and ankle rotational motions in the low-power mode. The sampling rate in the low-power mode was set to 3.125 Hz. Since the pace of an average person is two steps per second, a sampling rate of 3.125 Hz allows the detection of the vertical acceleration imparted by the impact force caused by the heel making contact with the ground at the beginning of the stance phase. As a result of comparing the waveforms of walking and ankle rotational motions, it was confirmed that the acceleration imparted by the impact force was much higher than the acceleration caused by the force of muscles for making ankle rotational motions. Based on this confirmation result, the first threshold value was set in consideration of individual differences. The setting of the first threshold value was verified from a histogram of the acceleration waveforms in the perpendicular direction during walking and ankle rotational motions.

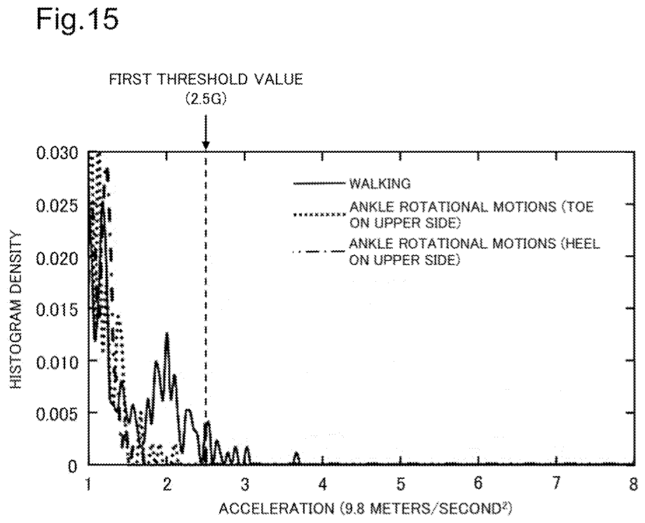

FIG. 15 illustrates a histogram of the acceleration waveforms in the perpendicular direction during walking and ankle rotational motions. In the acceleration waveform (solid line) imparted by the impact force, there is a section with acceleration of 2.5 times (2.5 G) the gravitational acceleration or more. In contrast to this, in the acceleration waveforms (the dotted line and the dashed-dotted line) imparted by ankle rotational motions, there is almost no section with acceleration equal to or more than 2.5 times the gravitational acceleration. This means that, if the threshold value is set to 2.5 times the gravitational acceleration, walking and non-walking can be distinguished from each other.

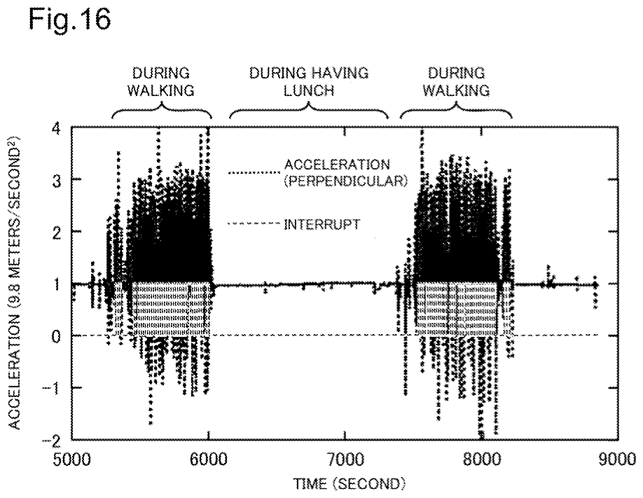

FIG. 16 illustrates a result of measuring the working status of the measurement device of the third example embodiment during walking and non-walking by making a test subject who is a company employee and goes out at lunch wear the measurement device and simulate daily life. During walking, the trigger signal (interrupt) was output to the control unit from the data acquisition unit, and the power consumption increased because the control unit set the data acquisition unit to the normal mode. In contrast to this, during having lunch, since the trigger signal was not output to the control unit from the data acquisition unit, the power consumption was exceptionally low because the data acquisition unit was not set to the normal mode.

A simulation was performed to detect walking after the data acquisition unit was activated, by analyzing walking waveforms. The data acquisition unit operating in the normal mode is assumed to record the gait data at a sampling rate of 50 Hz.

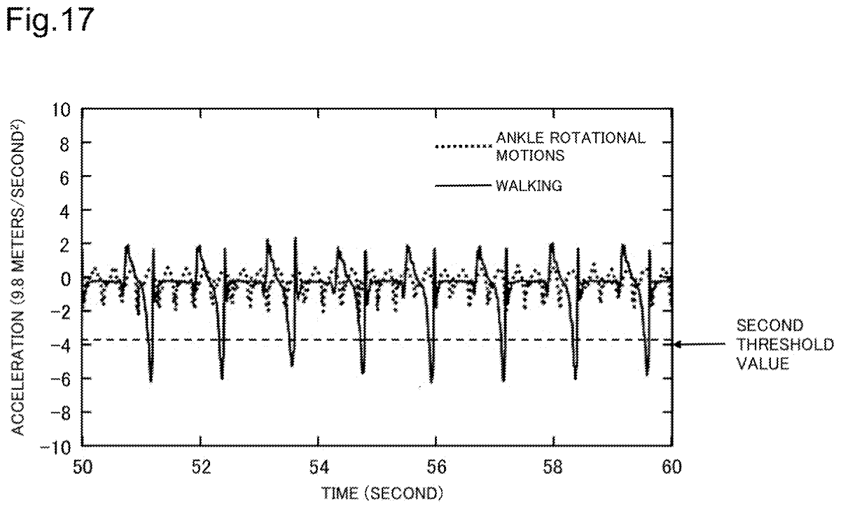

FIG. 17 illustrates acceleration waveforms (also referred to as walking waveforms) in the horizontal direction (positive in the forward direction) during walking and ankle rotational motions in the normal mode. In the walking waveform in FIG. 17, huge dips appear in the negative direction. These dips each represent acceleration in a direction opposite to the forward direction due to the sudden stop of the heel making contact with the ground at the beginning of the stance phase. Since these dips are observed only at the moment when the heel makes contact with the ground, these dips cannot be detected in a low sampling measurement in the low-power mode, but in the normal mode, the sudden stop during one step of the user is detected because the sampling rate is high. On the other hand, in the ankle rotational motions, the horizontal acceleration in the forward direction exerted by muscles is far less than the acceleration at the sudden stop. These characteristics can be utilized to set the second threshold value. That is, it could be detected that the user was walking, by setting the second threshold value for the dips according to the characteristics that the dips observed in FIG. 17 are generated only during walking. In contrast to this, when the dips are less than the second threshold value, it can be discriminated as a malfunction.

As described above, according to the present example, since the activation of the measurement device to the minimum extent was enabled by the two-stage waveform detection, it was confirmed that both of power saving and long life can be achieved.

(Hardware)

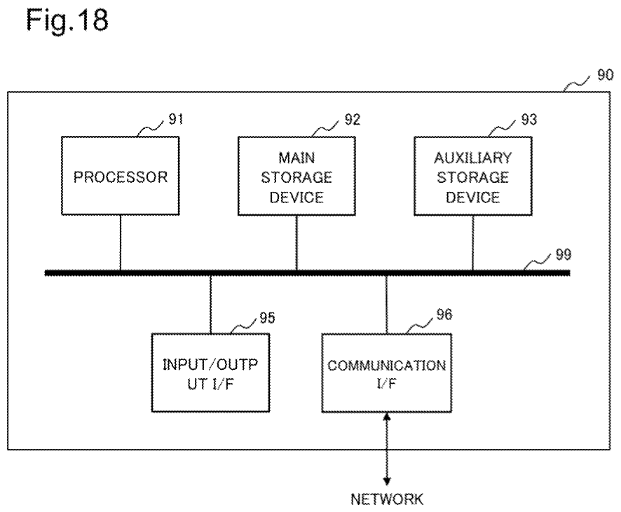

Here, the hardware configuration that executes the process of the measurement device according to each example embodiment of the present invention will be described with a computer 90 in FIG. 18 as an example. For example, the computer 90 can be configured as a microcomputer. The computer 90 illustrated in FIG. 18 is an example of a configuration for executing the process of the measurement device of each example embodiment, and does not limit the scope of the present invention.

As illustrated in FIG. 18, the computer 90 includes a processor 91, a main storage device 92, an auxiliary storage device 93, an input/output interface 95, and a communication interface 96. In FIG. 18, the interface is denoted as I/F as an abbreviation. The processor 91, the main storage device 92, the auxiliary storage device 93, the input/output interface 95, and the communication interface 96 are connected to each other via a bus 99 in such a way as to enable data communication. The processor 91, the main storage device 92, the auxiliary storage device 93, and the input/output interface 95 are connected to a network such as the Internet or an intranet via the communication interface 96.

The processor 91 expands programs stored in the auxiliary storage device 93 and the like into the main storage device 92, and executes the expanded programs. The present example embodiment can employ a configuration using a software program installed in the computer 90. The processor 91 executes processes by the measurement devices according to the present example embodiments.

The main storage device 92 has an area in which a program is expanded. The main storage device 92 can be, for example, a volatile memory such as a dynamic random access memory (DRAM). A nonvolatile memory such as a magnetoresistive random access memory (MRAM) may be configured and added as the main storage device 92.

The auxiliary storage device 93 stores diverse kinds of data. The auxiliary storage device 93 is constituted by a local disk such as a hard disk or a flash memory. A configuration for storing diverse kinds of data in the main storage device 92 can be employed in such a way that the auxiliary storage device 93 is omitted.

The input/output interface 95 is an interface for connecting the computer 90 and peripheral equipment. The communication interface 96 is an interface for connecting to an external system or device through a network such as the Internet or an intranet in accordance with a standard or specifications. The input/output interface 95 and the communication interface 96 may be commonly used as an interface for connecting to external equipment.

The above is an example of a hardware configuration for enabling the measurement device according to each example embodiment of the present invention. The hardware configuration in FIG. 18 is an example of a hardware configuration for executing the arithmetic process of the measurement device according to each example embodiment, and does not limit the scope of the present invention. A program for causing a computer to execute a process relating to the measurement device according to each example embodiment is also included in the scope of the present invention. Furthermore, a program recording medium on which a program according to each example embodiment is recorded is also included in the scope of the present invention.

The constituent elements of the measurement device of each example embodiment can be freely combined. The constituent elements of the measurement device of each example embodiment may be achieved by software or by a circuit.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

### REFERENCE SIGNS LIST

10, 20, 30 measurement device
11, 21, 31 data acquisition unit
12, 22, 32 control unit
33 learning unit
34 threshold value adjustment unit
111 acceleration sensor
112 angular velocity sensor
113 determination unit
114 data transmission unit
121 signal reception unit
122 activation unit
123 mode switching unit

What is claimed is:

1. A measurement device comprising:
a data acquirer configured to
measure a sensor detection value by a sensor in at least two operation modes including a first mode with low power and a second mode for operating at a high speed,
transmit a trigger signal in response to the sensor detection value exceeding a first threshold value while operating in the first mode,
transmit a first notification signal notifying that a user has started walking in response to the sensor detection value having exceeded a second threshold value a prescribed number or more of times during a prescribed period of time while operating in the second mode, and
start measuring gait data including a walking characteristic of the user wearing the sensor based on the sensor detection value; and
a controller comprising:
at least one memory storing instructions; and
at least one processor connected to the at least one memory and configured to execute the instructions to
switch the operation mode of the data acquirer to the second mode in response to receiving the trigger signal, and
switch the operation mode of the data acquirer to the first mode in response to a predetermined condition being satisfied after reception of the first notification signal.

2. The measurement device according to claim 1, wherein
the data acquirer includes:
an acceleration sensor that is configured to detect acceleration; and
an angular velocity sensor that is configured to detect angular velocity, wherein
the at least one processor is configured to execute the instructions to
operate either one of the acceleration sensor and the angular velocity sensor with low power consumption and stops operation of another of the acceleration sensor and the angular velocity sensor in the first mode, and
operate both of the acceleration sensor and the angular velocity sensor at a high speed in the second mode.

3. The measurement device according to claim 1, wherein
the at least one processor is configured to be activated in response to receiving the trigger signal,
execute the instructions to switch the operation mode of the data acquirer to the second mode, and
execute the instructions to switch the operation mode of the data acquirer to the first mode in response to the predetermined condition being satisfied after receiving the first notification signal, and
be shifted to a dormant state.

4. The measurement device according to claim 1, wherein
the controller is configured to be not activated in response to receiving the trigger signal, in a case where a predetermined period has not elapsed since last switching of the operation mode of the data acquirer.

5. The measurement device according to claim 1, wherein
the at least one processor is configured to execute the instructions to
switch the operation mode of the data acquirer to the first mode from the second mode at a stage in response to a predetermined period of time having elapsed since the first notification signal was received.

6. The measurement device according to claim 1, wherein
the data acquirer is configured to
transmit a second notification signal notifying that a malfunction has occurred, to the controller, when the sensor detection value has not exceeded the second threshold value the prescribed number or more of times during the prescribed period of time while operating in the second mode, and
the at least one processor is configured to execute the instructions to
switch the operation mode of the data acquirer to the first mode from the second mode in response to receiving the second notification signal.

7. The measurement device according to claim 1, wherein the at least one processor is configured to execute the instructions to:
generate a learning model for adjusting the first threshold value and the second threshold value by recording the first threshold value and the second threshold value in a log, and input the recorded log to a learner to; and
adjust the first threshold value and the second threshold value used by the data acquirer, by using the learning model, wherein
the data acquirer is configured to
transmit a second notification signal notifying that a malfunction has occurred, to, in response to the sensor detection value having not exceeded the second threshold value the prescribed number or more of times during the prescribed period of time while operating in the second mode, and
transmit the first threshold value and the second threshold value at that time,
the at least one processor is configured to execute the instructions to
switch, in response to receiving the second notification signal, the operation mode of the data acquirer to the first mode from the second mode, and
input a log including the first threshold value and the second threshold value newly received from the data acquirer, to the learner to generate the learning model.

8. The measurement device according to claim 1, wherein
the data acquirer is configured to
transmit the first notification signal notifying that the user wearing the sensor has started walking, in response to the sensor detection value having fallen below the second threshold value the prescribed number or more of times during the prescribed period of time while operating in the second mode.

9. A control method implemented by a measurement device that measures a sensor detection value in at least two operation modes including a first mode with low power and a second mode for operating at a high speed, the control method comprising:
outputting a trigger signal in response to the sensor detection value exceeding a first threshold value while operating in the first mode;
switching the operation mode to the second mode in response to the trigger signal;
generating a first notification signal notifying that a user wearing the sensor has started walking, in response to the sensor detection value having exceeded a second threshold value a prescribed number or more of times during a prescribed period of time while operating in the second mode;
starting measuring gait data including a walking characteristic of the user based on the sensor detection value; and
switching the operation mode to the first mode in response to a predetermined condition being satisfied after generation of the first notification signal.

10. A non-transitory program recording medium recorded with a program for operating a measurement device that measures a sensor detection value in at least two operation modes including a first mode with low power and a second mode for operating at a high speed, the program causing a computer to execute:
a process of outputting a trigger signal in response to the sensor detection value exceeding a first threshold value while operating in the first mode;
a process of switching the operation mode to the second mode in response to the trigger signal;
a process of generating a first notification signal notifying that a user wearing the sensor has started walking, in response to the sensor detection value having exceeded a second threshold value a prescribed number or more of times during a prescribed period of time while operating in the second mode;
a process of starting measuring gait data including a walking characteristic of the user based on the sensor detection value; and
a process of switching the operation mode to the first mode in response to a predetermined condition being satisfied after generation of the first notification signal.
